(19) United States
(12) Patent Application Publication
(10) Pub. No.: US 2007/0098779 A1
(43) Pub. Date: May 3, 2007

(54) Films and capsules made from modified carboxymethylcellulose materials

(76) Inventors: Andries Hanzen, Ede (NL); Henrica Wilhelmina Cornelia Vaessen-van Hoven, Voorthurizen (NL); Anja Maria Christina Petronella Hopman, Velp (NL); Ross Clark, San Diego, CA (US)

Correspondence Address: J M HUBER CORPORATION, 333 THORNALL STREET, PATENT DEPARTMENT, EDISON, NJ, 08837-2220, US

(21) Appl. No.: 11/264,260

(22) Filed: Nov. 1, 2005

(51) Int. Cl. A61K 9/48

(52) U.S. Cl. 424/451

(57) ABSTRACT

Films and/or capsules for the delivery of and/or coating of active ingredients are provided. Such edible films and/or capsules comprise particular modified carboxymethylcellulose (CMC) materials either alone or in combination with other types of hydrocolloids or biogums. The utilization of such modified CMC products aids in the production of such films and/or capsules through the availability of larger amounts of base materials with lower amounts of water requiring evaporation therefrom. In such a manner, not only may dimensionally stable, flexible, non-tacky, salt tolerant, and quick dissolving edible films and/or capsules be produced, but the amount of time required for such manufacture is minimal when compared with traditional methods of production with cellulosic-based materials. Furthermore, such novel edible films and/or capsules exhibit excellent clarity, retention of actives, and other physical properties (such as tensile strength, elongation, and ability to be cut into various shapes and sizes, etc.) that make such ultimate products attractive for use in a variety of functions. Furthermore, such films and/or capsules also exhibit properties in dissolution that permit controlled release of actives at any particularly desired rate. The novel method of manufacture as well as the ultimate edible films and/or capsules exhibiting such physical characteristics are also encompassed within this invention.

### FIELD OF THE INVENTION

This invention relates to edible films and/or capsules for the delivery of and/or coating of active ingredients. Such edible films and/or capsules comprise particular molecular weight-modified carboxymethylcellulose (CMC) materials either alone or in combination with other types of hydrocolloids, biogums, cellulose ethers, and the like. The utilization of such modified CMC products aids in the production of such films and/or capsules through the availability of larger amounts of base materials with lower amounts of water requiring evaporation therefrom. In such a manner, not only may dimensionally stable, flexible, non-tacky, salt tolerant, and quick dissolving edible films and/or capsules be produced, but the amount of time required for such manufacture is minimal when compared with traditional methods of production with cellulosic-based materials. Furthermore, such novel edible non-digestible films and/or capsules exhibit excellent clarity, retention of actives, and other physical properties (such as tensile strength, elongation, and ability to be cut into various shapes and sizes, etc.) that make such ultimate products attractive for use in a variety of functions. Furthermore, such films and/or capsules also exhibit properties in dissolution that permit controlled release of actives at any particularly desired rate. The novel method of film manufacture as well as the ultimate edible films and/or capsules exhibiting such physical characteristics are also encompassed within this invention.

### BACKGROUND OF THE INVENTION

Films and capsules, particularly of the edible variety, have been popular for the delivery of active ingredients such as pharmaceuticals, breath fresheners, oral care materials, foodstuffs, and other like products for ingestion within a person's oral cavity. Furthermore, such films are utilized within coatings, seals, and other like objects for such materials as dyes, deodorants, detergents, tablets, and the like. Flexible capsules have been utilized for pharmaceutical delivery for some time now and have proven to be invaluable, particularly for patients that exhibit difficulty in swallowing pills. Of more recent development have been films that permit delivery of certain actives (such as, as noted above, breath fresheners, and the like) through the rapid dissolution thereof within the mouth of a user with concomitant absorption or other like action by the active after the film is removed through exposure to sufficient moisture. As other active delivery systems (chewing gum, lozenges, etc.) exhibit certain drawbacks in comparison, such films have increased in usage in recent years.

Such films (of the edible variety) are generally comprised of non-toxic ingredients that permit the desirable properties of quick dissolution, flexible film production, and dimensional stability for proper cutting into specific shapes and sizes. Typical films of this type include pullulan, cellulosics (such as hydroxypropylmethyl cellulose), carrageenan, pectin, as well as mixtures of certain low molecular weight varieties of products and high molecular weight types. Although such films have been produced in large-scale methods over the last few years, there are certain limitations that are either aesthetically questionable to the consumer or include increased manufacturing costs that are passed on to the same person ultimately.

For example, clarity and low tackiness are generally properties sought after by the consumer. Clear, transparent films give an appearance of uniformity and order, whereas the utilization of a tacky film will most likely result in a film that will dissolve only after sticking to the user's palate for an extended period of time. Furthermore, tackiness may also lend itself to packed films that adhere to one another, thus increasing the likelihood of simultaneous use of multiple films or damage to films during removal from the packaging in which such products are stored. Thus, low tackiness is desirable for such film products.

Additionally, costs of manufacture have proven difficult to reduce for such films, particularly when the amount of film-forming component is relatively low. Solutions of, for instance, hydroxypropylmethyl cellulose (HPMC) including an excess of about 80% or higher by weight of water are typical for such film-forming materials. Once the solution is spread on a suitable plate and smoothed (such as by a blade) to a substantially uniform thickness, the time required to effectively form the desired film is dependent upon the humidity of the environment as well as the amount of water required to be evaporated. At such a high level of water, the needed evaporation time is excessive or the amount of heat needed to effectuate such evaporation quickly increases the manufacturing costs to a rather high level. A decrease in water within the initial solution, although, it may reduce evaporation time ultimately, leads to other problems, most notably the necessity for sufficient mixing to thoroughly disperse the cellulosic materials throughout the solution for proper uniform film production. As such, with too little water present, the amount of time and effort required for such needed thorough mixing is inordinately high. In either situation, the cost of manufacture is impacted by the amount of water needed and the ultimate cost for such film production is ultimately passed on to the consumer.

Thus, there has been an aim to provide edible films and/or capsules for like delivery of actives that exhibit the same properties, at least, at lower cost of production.

The closest prior art teaches edible, consumable films for the delivery of certain actives, such as flavoring and/or breath freshening agents, that are formulated to dissolve in the user's oral cavity. Such prior art includes films made from water soluble polymer such as pullulan or hydroxypropylmethyl cellulose and an essential oil selected from thymol, methyl salicylate, eucalyptol and/or menthol; film compositions containing therapeutic and/or breath freshening agents, prepared from water soluble polymers such as hydroxypropylmethyl cellulose, hydroxypropylcellulose, etc., and a polyalcohol (such as polyglycols); as well as consumable films that comprise hydroxyalkylmethylcellulose, pre-gelatinized starch, and a flavoring agent.

Other teachings exist that concern the utilization of cellulosic-based polymers for film production; however, in each instance, the specific teaching pertains to non-modified (typical high molecular weight range) starting materials. As such, the films made therefrom, although they may exhibit effective properties for the purposes for which they are made, suffer from high production costs, high complexity in manufacture, particularly as it concerns the requirement of initially providing a thoroughly mixed solution prior to film creation, as well as difficulty in ensuring all of the water within the initially produced solution is properly evaporated during production. Relative humidity may pose a problem for such films and/or capsules during production as well as thereafter (such as during shelf storage), and polysaccharides, such as CMC, hydroxypropylmethylcellulose, and the like, all seem to suffer certain drawbacks as a result of water content, not to mention the presence of too much salt within the target environment. Thus, there remains a definite interest in providing the industry a film and /or capsule that is relatively simple to manufacture, requires very little mixing and/or evaporation of water during production, exhibits excellent flexibility, dimensional stability, and active retention, and will dissolve quickly within the target location for efficient and effective delivery of the desired active. As such, to date, there is a lack of teaching or fair suggestion of any such films and/or capsules, particularly any such products that comprise molecular weight-modified CMC materials. With such in mind, it has now been determined that such beneficial films and/or capsules are available through the utilization of particularly selected CMC starting materials, as well as combinations of such materials with other polysaccharides and/or biogums.

### BRIEF DESCRIPTION OF THE INVENTION

Accordingly, it is one advantage of the present invention to provide a low-complexity method of producing thin, non-toxic, clear films of high flexibility and quick dissolution in an aqueous environment. Another advantage of the present invention is to provide such a film and/or capsule material that exhibits such excellent properties as noted above, as well as effective and efficient delivery of actives incorporated therein.

Accordingly, this invention encompasses a novel film and/or capsule comprising modified CMC materials exhibiting a molecular weight range of from 1500 to 75000 and a degree of substitution of less than about 1.5. Furthermore, this invention encompasses a method of producing such a film and/or capsule comprising the steps of a) providing a CMC materials exhibiting a molecular weight range of from 80000 to 3000000 and degree of substitution of less than about 1.5; b) degrading said CMC materials by exposing said materials to an enzyme in an amount and for a period of time sufficient to reduce the molecular weight range of said CMC materials to a range of from 1500 to 75000; c) inactivating said enzyme; d) producing a solution of the resultant modified CMC materials of step “b” with at most 70% by weight of water and optionally including at most 12.5% of a plasticizer; and e) forming a film or capsule through proper application of said solution to a proper surface and allowing said water therein to evaporate therefrom. Such films thus exhibit at least the same film strength, rapid film dissolution, and delivery capabilities of active ingredients as previously made films and/or capsules, but with lower manufacturing costs, and potentially reduced tackiness as those currently utilized within the pertinent markets. Such an improvement has been realized through the utilization of a single modified CMC component as well, thereby permitting a reduction in manufacturing complexity of films. Such is a significant benefit over the comparative prior film compositions that have relied upon combinations of ingredient polymers to provide similarly effective films and/or capsules. Although a single modified CMC polymer may be utilized for this application, it is noted that combinations of the required modified CMC polymer with other polymeric additives, such as hydrocolloids, biogums, and cellulose ethers (either gel-forming or non-gelling viscosity building types, depending on the potential benefits desired from such an additive) may be practiced as well. Such a film and/or capsule, of the modified CMC alone or in combination with such other optional gel-forming or non-gelling viscosity building additives is thus highly desired from a cost perspective as well as quick and complete dissolution when exposed to sufficient moisture within the oral cavity. Such a specific characteristic is advantageous since undissolved film residue imparts an unacceptable, unpalatable, slimy feel to the palate of the user.

### SUMMARY OF THE INVENTION

For the purpose of this invention, the term “film” is intended to encompass a solid, flexible sheet of polymer material that has a very low ratio of thickness to area (width multiplied by length).

The term “capsule” is intended, for purpose of this invention, to encompass a flexible container that may be used to carry and active material into the digestive tract for later delivery of this active agent.

Polysaccharides, such as certain cellulosic-based types (carboxymethylcellulose, as one non-limiting example), have been utilized within numerous fields for many years as viscosity modifiers, carriers, anti-redeposition agents, and other like purposes within the paper, oil, food, paint, and detergent industries, to name a few. The benefits of modified cellulosics water-soluble polymers have been provided as well, particularly within U.S. Pat. No. 5,569,483 to Timonen et al., as it pertains to substitution of fat within foodstuffs, and within U.S. Pat. No. and 5,543,162 to Timonen et al., as it pertains to the utilization of such enzymatically modified cellulosics in combination with hydrophilic polymers (such as gelatin) in coacervation methods of forming capsules. There is no discussion within either of these references of the ability of specific modified CMC materials for the purpose of providing excellent film, capsule, or other type of coating, particularly those that meet certain molecular weight and thus viscosity requirements.

The present invention relates to an edible film composition comprising a safe and effective amount of at least a modified CMC material, optionally, a further amount of another polysaccharide or biogum material, optionally, a safe and effective amount of a plasticizing agent, and, a safe and effective amount of an ingredient, including, as examples, a flavoring agent, a pharmaceutical agent, an oral care additive, an anti-inflammatory agent, an antimicrobial agent, a surfactant, a sweetener, a vitamin, and the like. The films of this invention may be utilized as delivery systems for such active ingredients through dissolution within the oral cavity of a user and/or patient, or as a coating or seal for materials including, without limitation, foodstuffs, soaps, detergents, tablets, and the like, or potentially can be modified to form capsules for transport of active ingredients to the oral cavity of a user and/or patient (delivery of actives in capsules takes place in the stomach/gastro-intestinal system).

### DETAILED DESCRIPTION OF THE INVENTION

All percentages and ratios used hereinafter are by weight of total composition, unless otherwise indicated. As used herein, percentage by weight of the film composition means percent by weight of the wet film composition, unless otherwise indicated.

All U.S. patents cited herein are hereby incorporated in their entirety by reference.

The edible film and/or capsule compositions of the present invention comprise at least one molecular weight-modified CMC material. Although such degradation may be accomplished through any type of well known method, such as acid, radiation, oxidation and heat degradation, preferably the degradation step is provided through enzymatic exposure. Thus, the initial method step is actually providing the degrading CMC material for further use thereof. Such a step may be accomplished similarly to that taught within either of the Timonen et al. patents discussed above. In essence, a CMC having the desired degree of substitution and initial molecular weight is subjected to a preselected amount of cellulase enzyme in order to reduce the overall molecular weight of the CMC material itself to a level proper for film and/or capsule production. The CMC selected for this step, as alluded to above, must exhibit a proper degree of substitution (i.e., the average amount of carboxymethyl groups per glucose unit) in order to permit the ultimate generation of a film and/or capsule exhibiting the requisite characteristics of rapid dissolution, dimensional stability, and low tackiness, at least. For certain end uses, such as those involving ingestion as or in tandem with foodstuffs, the degree of substitution is preferably, though not necessarily, lower than about 0.95. For other types of end uses, higher levels may be permitted (such as up to about 1.5). The initial molecular weight may be within a broad range as long as the ultimate molecular weight range meets the requirements that lead to the same type of proper film and/or capsule generation in terms of the physical characteristics noted above. Thus, an initial molecular weight range, as measured as by using GPC analysis of from 80,000 to about 3,000,000 is acceptable. The thus preselected CMC starting material can then be exposed to an amount of cellulase that coincides, in combination with the amount of time of such exposure, pH and temperature with the ultimate degradation of the CMC material into individual strands thereof exhibiting a range of molecular weights from 1,500 to 75,000. If the molecular weight is too low (below 1,500), then the film or capsule will be too friable to properly function. Preferably, though not necessarily, the molecular weight will be between about 20,000 and 50,000 for the modified CMC materials. A lower molecular weight range (i.e., from 1,500 to about 20,000) may be utilized as well, but will preferably, though, again, not necessarily, be compensated for with a higher degree of substitution. After the time of enzyme exposure is completed, the cellulase can then be inactivated through heat exposure, as one example, thereby preventing further degradation of the CMC from occurring. The enzyme can be removed from within the modified CMC solution used for film and/or capsule production.

The molecular weight range sought after for the modified CMC materials transfers to a viscosity measurement for the solutions used to ultimately produced the target films typically within a range of 10,000 mPas to 45,000 mPas. It has been found as well that such viscosity measurements appear to contribute to the overall effectiveness of the ultimately formed films and/or capsules in combination with the degree of substitution of the starting CMC materials themselves. Thus, it has been determined that such molecular weight and viscosity properties are critical to the success of the overall invention, at least when the sole film-forming component of the solution is the modified CMC material.

As noted previously, one surprising result of this invention is that the modified CMC can be utilized as such a sole film-forming component. Most commercially available films require the utilization of combinations of different polymers to attain desired film properties; however, it has surprisingly been determined that the modified CMC polymers utilized within this invention are sufficient on their own to achieve such results. The ability to form a film and/or capsule that meets or exceeds the aforementioned physical characteristics as well as can withstand certain salt and relative humidity exposures without appreciably effecting the dimensional stability and usefulness of the ultimate end use product was unexpected. If desired, however, one may include other hydrocolloids, biogums, and/or cellulose ethers to provide increases in salt and/or humidity protection, or to provide viscosity build within film- and/or capsule-formulations, or to provide gel formation for the same types of formulations, and/or one may include a plasticizer in order to increase film flexibility or provide increases in dimensional stability and other physical characteristics of the subject films and/or capsules as well. Such a molecular weight-modified CMC polymer exhibits excellent compatibility with such other possible polymers and thus their optional presence should not be problematic.

The other types of optional polymeric additives that may be utilized within the inventive films and/or capsules, again, in addition to the required modified CMC materials, include, without limitation, non-gelling viscosity building additives selected from the group consisting of cellulose ethers, such as methyl cellulose, (non-modified) carboxymethylcellulose, hydroxyethylcellulose, hydroxypropylcellulose, hydroxypropylmethylcellulose, and mixtures thereof; biogums, such as xanthan gum, diutan gum, rhamsan gum and welan gum, gellan gum, and mixtures thereof; and hydrocolloids such as carrageenan, pectin, gum arabic, guar, locust bean gum, gum tragacanth, tara gum, sodium alginate, acacia gum, pullulan, pustulan, scleroglucan, and mixtures thereof; and any combinations or mixtures thereof such different types of hydrocolloids. Furthermore, other additives that impart gel-forming characteristics to the modified CMC formulations include, without limitation, gel-forming additives selected from the group consisting of of gellan gum (high and low acyl forms), carrageenan (kappa and iota types), xanthan/locust bean gum, sodium alginate, curdlan, MHPC, pectin, and any combinations or mixtures thereof. The optional polymeric additives listed above may be present therein in an amount of from 0.05 to 50% by weight of the entire film and/or capsule.

One benefit of utilizing the modified CMC, particularly, whether alone or in combination with these other types of hydrocolloids and/or biogums, is the reduced viscosity exhibited thereby permits greater amounts of the modified CMC to be introduced within the film-forming solution than is customary. As discussed above, this permits a reduction in the amount of water needed for a proper film-forming composition to be produced and drastically reduces the time required for water evaporation. Furthermore, the film-forming solution can be easily and thoroughly mixed under relatively low energy levels such that a properly dispersed solution is accorded the film producer as well. The modified CMC materials are present as long strands, rather than as coiled globules of CMC; thus, the avoidance of detrimental lumps within the film-forming solution is possible at the aforementioned low energy mixing levels. The proper film-forming solutions thus will comprise from about 10 to about 50% of the modified CMC, from about 50 to about 90% by weight of water, and optionally, from 0 to about 12.5% by weight of a plasticizer. The active ingredient is also incorporated within the film-forming solution and is thoroughly mixed therein as well for proper dispersion within the ultimate film. Such an additive may be present in an amount of from about 0.001 to about 70% by weight of the entire composition.

In addition to the above essential modified CMC film-forming agents, the solution may also comprise other additional film-forming agents other than the hydrocolloids, cellulose ethers, and/or biogums listed above, such as, without limitation, polyvinyl pyrrolidone, polyvinyl alcohol, sodium alginate, polyethylene glycol, polyacrylic acid, methylmethacrylate copolymer, carboxyvinyl polymer, starch, amylose, high amylose starch, hydroxypropylated high amylose starch, dextrin, , chitin, chitosan, levan, elsinan, collagen, gelatin, zein, gluten, soy protein isolate, whey protein isolate, casein, and mixtures thereof.

The compositions of the present invention also comprise a safe and effective amount of a plasticizing agent to improve flexibility and reduce brittleness of the edible film composition. Suitable plasticizing agents of the present invention include, but are not limited to, polyols (such as sorbitol; glycerin; polyethylene glycol; propylene glycol; acetylated monoglyceride; hydrogenated starch hydrolysates; corn syrups; and derivatives thereof; xylitol; glycerol monoesters with fatty acids; triacetin; diacetin; and monoacetin) and mixtures thereof. In one embodiment the plasticizing agent of the present invention is glycerol.

The compositions of the present invention may also comprise a safe and effective amount of an additive selected from the group consisting of a flavoring agent, an antimicrobial agent, an oral care and/or a pharmaceutical agent, a surfactant, a sweetener, a nutrient (such as a vitamin or mineral), and any combinations thereof.

Suitable flavoring agents include any well known food flavoring (of which there are a vast variety to choose from) including, without limitation, examples such as oil of wintergreen, oil of peppermint, oil of spearmint, clove bud oil, menthol, eucalyptol, lemon, orange, cinnamon, vanillin, and the like, and mixtures thereof. In another embodiment, in order to stabilize the flavor, the compositions may optionally comprise a vegetable oil.

The present invention may optionally comprise a safe and effective amount of an oral care active agent and/or a pharmaceutical active agent. The oral care active agent suitable for use herein is selected from the group consisting of anticalculus agent, fluoride ion source, antimicrobial agents, dentinal desensitizing agents, anesthetic agents, antifungal agents, anti-inflammatory agents, selective H-2 antagonists, anticaries agents, nutrients, and mixtures thereof. The oral care active agent preferably contains an active at a level where upon directed use, the benefit sought by the user is promoted without detriment to the oral surface to which it is applied. Examples of the “oral conditions” these actives address include, but, are not limited to, appearance and structural changes to teeth, whitening, stain removal, plaque removal, tartar removal, cavity prevention and treatment, inflamed and/or bleeding gums, mucosal wounds, lesions, ulcers, aphthous ulcers, cold sores, tooth abscesses, and the elimination of mouth malodor resulting from the conditions above and other causes such as microbial proliferation. Suitable oral care actives include any material that is generally considered safe for use in the oral cavity and that provides changes to the overall appearance and/or health of the oral cavity. The level of oral care substance in the compositions of the present invention is generally, unless specifically noted, from about 0.01% to about 50%, preferably from about 0.1% to about 20%, more preferably from about 0.5% to about 10%, and even more preferably from about 1% to about 7%, by weight of the dry film composition.

The anticaries agent may be selected from the group consisting of xylitol, fluoride ion source, and mixtures thereof. The fluoride ion source provides free fluoride ion during the use of the composition. In one embodiment the oral care active agent is a fluoride ion source selected from the group consisting of sodium fluoride, stannous fluoride, indium fluoride, organic fluorides such as amine fluorides and sodium monofluorophosphate. Sodium fluoride is the fluoride ion in another embodiment. In one embodiment the anticalculus agent is selected from the group consisting of polyphosphates and salts thereof; diphosphonates and salts thereof; and mixtures thereof. In another embodiment the anticalculus agent is selected from the group consisting of pyrophosphate, polyphosphate, and mixtures thereof.

The anticalculus agent is a polyphosphate, understood to mean a compound consisting of two or more phosphate molecules arranged primarily in a linear configuration, although some cyclic derivatives may be present. Counterions for these phosphates may be the alkali metal, alkaline earth metal, ammonium, C₂-C₆ alkanolammonium and salt mixtures. Polyphosphates are generally employed as their wholly or partially neutralized water soluble alkali metal salts such as potassium, sodium, ammonium salts, and mixtures thereof. The inorganic polyphosphate salts include alkali metal (e.g. sodium) tripolyphosphate, tetrapolyphosphate, dialkyl metal (e.g. disodium) diacid, trialkyl metal (e.g. trisodium) monoacid, potassium hydrogen phosphate, sodium hydrogen phosphate, and alkali metal (e.g. sodium) hexametaphosphate, and mixtures thereof. Polyphosphates larger than tetrapolyphosphate usually occur as amorphous glassy materials. In one embodiment the polyphosphates are those manufactured by FMC Corporation which are commercially known as Sodaphos, Hexaphos, and Glass H, and mixtures thereof.

Pyrophosphate salts may be utilized in a like manner to the polyphosphates noted above. Such would include alkali metal pyrophosphates, di-, tri-, and mono-potassium or sodium pyrophosphates, dialkali metal pyrophosphate salts, tetraalkali metal pyrophosphate salts, and mixtures thereof. More specifically, these may be, in non- limiting fashion, trisodium pyrophosphate, disodium dihydrogen pyrophosphate, dipotassium pyrophosphate, tetrasodium pyrophosphate, tetrapotassium pyrophosphate, and mixtures thereof. Optional agents to be used in place of or in combination with the pyrophosphate salt include such known materials as synthetic anionic polymers, including polyacrylates and copolymers of maleic anhydride or acid and methyl vinyl ether (e.g., Gantrez), as described, for example, in U.S. Pat. No. 4,627,977, to Gaffar et al., the disclosure of which is incorporated herein by reference in its entirety; as well as, e.g., polyamino propoane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof.

Antimicrobial antiplaque agents may also by optionally present in the present compositions. Such agents may include, but are not limited to, triclosan, 5-chloro-2-(2,4-dichlorophenoxy)-phenol, chlorhexidine, alexidine, hexetidine, sanguinarine, benzalkonium chloride, salicylanilide, domiphen bromide, cetylpyridinium chloride (CPC), tetradecylpyridinium chloride (TPC), N-tetradecyl-4-ethylpyridinium chloride (TDEPC), octenidine, delmopinol, octapinol, and other piperidino derivatives; effective antimicrobial amounts of essential oils and combinations thereof for example citral, geranial, and combinations of menthol, eucalyptol, thymol and methyl salicylate; antimicrobial metals and salts thereof for example those providing zinc ions, stannous ions, copper ions, and/or mixtures thereof; bisbiguanides, or phenolics; antibiotics such as augmentin, amoxicillin, tetracycline, doxycycline, minocycline, and metronidazole; and analogs and salts of the above antimicrobial antiplaque agents; anti-fungals such as those for the treatment of candida albicans.

Anti-inflammatory agents may also be present in the oral compositions of the present invention. Such agents may include, but are not limited to, non-steroidal anti-inflammatory agents such as aspirin, ketorolac, flurbiprofen sodium, ibuprofen, acetaminophen, diflunisal, fenoprofen calcium, naproxen, indomethacin, ketoprofen, tolmetin sodium, piroxicam and meclofenamic acid, COX-2 inhibitors such as valdecoxib, celecoxib and rofecoxib, and mixtures thereof.

The present invention may also include a safe and effective amount of a selective H-2 antagonist such as, without limitation, cimetidine, etintidine, ranitidine, tiotidine, lupitidine, donetidine, famotidine, roxatidine, pifatidine, lamtidine, zaltidine, nizatidine, mifentidine, ramixotidine, loxtidine, bisfentidine, sufotidine, ebrotidine, and impromidine.

Nutrients include minerals, vitamins, oral nutritional supplements, enteral nutritional supplements, and mixtures thereof. Minerals that can be included with the compositions of the present invention include calcium, phosphorus, fluoride, zinc, manganese, potassium and mixtures thereof. Vitamins can be included with minerals or used separately. Vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Oral nutritional supplements include amino acids, lipotropics, fish oil, and mixtures thereof. Amino acids include, but, are not limited to L-Tryptophan, L-Lysine, Methionine, Threonine, Levocamitine or L-carnitine and mixtures thereof. Lipotropics include, but, are not limited to choline, inositol, betaine, linoleic acid, linolenic acid, and mixtures thereof. Fish oil contains large amounts of Omega-3 polyunsaturated fatty acids, eicosapentaenoic acid and docosahexaenoic acid. Antioxidants that may be included in the oral care composition or substance of the present invention include, but are not limited to Vitamin E, ascorbic acid, Uric acid, carotenoids, Vitamin A, flavonoids and polyphenols, herbal antioxidants, melatonin, aminoindoles, lipoic acids and mixtures thereof. Enteral nutritional supplements include, but, are not limited to protein products, glucose polymers, corn oil, safflower oil, and medium chain triglycerides.

Anti-pain or desensitizing agents and anesthetic agents can also be present in the oral care compositions or substances of the present invention. Such agents may include, but are not limited to, strontium chloride, potassium nitrate, natural herbs such as gall nut, Asarum, Cubebin, Galanga, scutellaria, Liangmianzhen, Baizhi, etc. Anesthetic agents include lidocaine, benzocaine, etc.

The pharmaceutical active agent suitable for use herein is selected from the group consisting of sedatives, hypnotics, antibiotics, antitussives, antihistamines, non-sedating antihistamines, decongestants, expectorants, mucolytics, antidiarrheals, analgesics-antipyretics, proton pump inhibitors, general nonselective CNS stimulants, drugs that selectively modify CNS function, antiparkinsonism drugs, narcotic-analgesics, psychopharmacological drugs, laxatives, dimenhydrinates, and mixtures thereof. Preferred pharmaceutical actives suitable for use as an active ingredient herein include antitussives, antihistamines, non-sedating antihistamines, decongestants, expectorants, mucolytics, analgesics-antipyretics, anti-inflammatory agents, antidiarrheals, and mixtures thereof.

Suitable surfactants are those which are reasonably stable and include nonionic, anionic, amphoteric, cationic, zwitterionic, synthetic detergents, and mixtures thereof.

The present compositions may optionally comprise sweetening agents including sucralose, sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame and cyclamate salts, especially sodium cyclamate and sodium saccharin, and mixtures thereof.

Coolants, salivating agents, warming agents, coloring agents, and numbing agents can be used as optional ingredients in compositions of the present invention as well.

### PREFERRED EMBODIMENTS OF THE INVENTION

The film compositions utilized in accordance with the invention are formed by processes conventional in the arts, e.g. the paper-making and/or film making industries. Generally the separate components of the film are blended in a mixing tank until a homogeneous mixture is achieved. Thereafter, the films can be cast to an acceptable thickness, on an appropriate substrate. Examples of such substrates include Mylar, continuous moving stainless steel belt (eventually entering a dryer section), release paper and the like. The webs are then dried, e.g. in a forced-air oven. The temperature of the drying air and length of drying time depend on the nature of the solvent utilized as is recognized in the art. Most of the films contemplated herein, however, are dried at a temperature between about 25° C. (i.e., ambient temperature) and 140° C. (with a lower temperature preferred to reduce costs), for a duration of about 20 minutes to about 60 minutes, in another embodiment from about 30 to about 40 minutes. Drying of these films should be carried out in a way that moisture gradients are minimized in the film. Such gradients come from rapid drying and lead to curling and lack of dimensional stability. When dried properly, the films will have a final water activity of 0.5 (+/−0.25) so that they do not either take up or lose significant amount of water when exposed to normal ambient conditions. The moisture content will vary depending upon the composition of the film, it's water activity rather than water content that is the parameter to be controlled. Films with a low water content may be dried in as little as 30 minutes at 40° C. The optimal temperature of the film during drying is usually lower than 65 C.°. Higher temperatures can be used, especially if the film is dried simultaneously from the top and bottom. This can be accomplished by using a heated metal belt of the bottom and indirect intra-red heating from above. Microwave techniques and other novel drying technologies can also be used successfully. After exiting from the dryer section of the casting belt, the film can be wound on a spool for storage under sanitary conditions. The film can be slit into two inch rolls for further cutting to form 1 inch by 2 inch (or other desired dimensions) and then stacked and subsequently individually packaged.

Extrusion is also a possible method of film manufacture. The mechanical particulars of the extrusion process, e.g. the particular equipment utilized, the extruding force, the shape and temperature of the orifice are considered to be within the skill of the art and can be varied in a known manner to achieve the physical characteristics of the films described herein.

The films herein are generally between about 1 and about 10 mils (about 0.025 mm to about 0.25 mm), in another embodiment are from about 1.2 to about 2.5 mils (about 0.03 mm to about 0.064 mm) thick. A convenient width for such films is about 0.75 to about 1 inch, although the width of the film is not particularly critical to the practice of the invention. The film can be produced in any length. However, in view of the fact that the novel dosage forms produced in accordance with the invention are suited to high speed manufacture, the films should be prepared in large quantity, e.g. 15,000 feet or more which can be stored, e.g. on cores or spools.

Likewise, the capsules made therefrom may be produced through typical capsule-making procedures utilizing the same basic solutions as the film-making methods. The required modified CMC may be applied in hard (two-piece) and/or soft (one-piece) capsules. The term “hard capsules” connotes that such materials must retain their shape from the time of manufacture through being filled and ultimately until they are ingested for use. “Soft capsules”, however, exhibit a soft shell only at the moment they are formed and filled. One-piece capsules are generally sold as formulated products whereas hard capsules are generally manufactured empty and filled at a later time.

Gelatin has traditionally been the material of choice within the capsule industry. Gelatin exhibits a number of properties that make such a material a proper candidate for capsule manufacture including good film forming properties (strength and flexibility, primarily), good solubility in biological fluids at typical body temperature, low viscosity at 50° C. at high solids concentrations, and a gel state at low temperatures. Likewise, methylhydropropyl cellulose has recently found favor within the capsule industry for the same basic reasons.

Soft capsules containing gelatin are made by passing two flexible sheets of gelled plasticized gelatin solution between a pair of rotating cylinders. The gelatin sheets are passed over and are sealed together by mechanical pressure and heat. The films are half sealed before the filling process starts. The cylinders have cavities in their surfaces and the gelatin sheet is forced into their shape by the pressure of the fill material as it is pumped between the cylinders. Subsequent to such a step, the resultant capsules are then dried. As alluded to above, the gelatin solution requires a significant quantity of plasticizer to form the necessary flexible sheets for introduction within the capsule-production process.

Hard capsules containing gelatin are made by dipping ‘cold’ stainless steel mold pins at a temperature of 22° C. in a 30-40% gelatin solution present at 50-60° C. The pins will pick up the target gelatin due to gelling while the excess runs off. The viscosity of the gelatin solution determines the quantity picked up by the molds during capsule formation. The pin bars are then rotated in order to facilitate spread of the gelatin as uniformly as possible over the subject mold surface. As before, the last step is a drying step.

Hard capsules containing MHPC are made on smaller mould pins to enable capsules with thinner walls to be made. This is required to give them sufficient strength to be filled and retain the same external dimensions as a gelatin capsule. Two methods are used. One is using thermal gelation of HPMC. The other is using a gelling agent (e.g. carrageenan or gellan gum) and a gelation promoter.

As the inventive modified CMC capsules do not exhibit the thermal gelation behavior that MHPC or gelatin shows, typically a gelling agent should be added. In case of soft capsules this is required to form a wet sheet together with the modified CMC, that will be mechanically deformable. When applied as hard capsules the gelling agent is needed to get sufficient surface gelling at the mold pins thereby picking up sufficient modified CMC material to form the required (uniform) capsule dimension.

The gelling agent should not compromise the modified CMC film-forming properties, nor cause too great a viscosity increase within the solution.

The final modified CMC-containing capsule films should have a suitable strength. Furthermore, the capsules should be readily soluble in biological fluids at body temperature.

An example where the modified CMC can be used to form capsules would be in combination with gellan gum. The low acyl version of this product has a setting temperature of approximately 40° C. (within the same range as gelatin). However, the gellan gum is much higher in gel strength than commonly used gelatins and so only a low concentration can be used. Higher levels result in gellan gum-alone solutions that are too thick and viscous to be processed. Using the modified CMC in conjunction with gellan gum allows the CMC to function as a film former and the gellan gum to thermally set and form the capsule in a manner similar to the gelatin. Other gelling hydrocolloids can function in the same way with the only requirement being that they have an easily triggered gel mechanism. Those skilled in the art of hydrocolloids are familiar with the thermal gelation of xanthan and locust bean gums or with the calcium gelation mechanism of sodium alginate, and such possible alternatives are thus non-limiting examples of potential gelling hydrocolloids for this purpose. A wide range of gelling hydrocolloids can be used in conjunction with the modified CMC when it is realized that the film forming properties of the modified CMC can be effectively paired with the gel forming properties of the second hydrocolloid system. Thus, there exists a wide range of possibilities with respect to unique capsule formulations in combination with the inventive modified CMC materials.

Such approaches (using a gel forming system with some other material of low viscosity) have been used before but these systems do not take advantage of the film forming properties of the modified CMC described in this invention. Since a capsule is a special case of film formation, the use of modified CMC of reduced molecular weight provides a significant improvement over capsules previously produced with gelling hydrocolloids in combination with simple “fillers” (such as maltodextrose). In this situation, then, this invention concerns, as one possible embodiment, the instance whereupon the non-gelling hydrocolloid serves an important role as a film former for capsule formation.

The processes followed for production of the inventive modified CMC materials and films and/or capsules made therefrom are delineated below.

1. Modified CMC Production

Initially, samples of different CMC materials were modified to different levels of molecular weights in order to provide materials for ultimate film production. In each instance, the basic degradation method was preferably performed enzymatically and followed the basic steps of: Tap water was charged to a barrel that was placed in a water bath of 50° C. From a food grade cellulase (Econase CE from AB enzymes) from Trichoderma reesei, 0.1-1% (weight percent on dry CMC basis) was added to the water (exhibiting a pH of 5.8 as adjusted by a 21% phosphoric acid solution). While stirring thoroughly CMC from CPKelco (the different types are noted within Table 1, below) was slowly added over a period of an hour to a concentration of 20% in water. The pH was then adjusted again to 5.8 using the same phosphoric acid solution. The reaction was performed at 50° C. while stirring for 16 hours and was eventually stopped by inactivating the enzyme in an autoclave at 121° C. for one hour. The resultant modified CMC solutions were then dried by either freeze-drying or spray drying.

TABLE 1
Characteristics of Modified CMCs

| | CMC starting | Mod CMC | Mod CMC | Enzyme |
|---|---|---|---|---|
| | material | Deg of | Mol. | Amt. |
| Ex. | Tradename | Subst. | Weight | (% b/w) |
| 1 | CEKOL ® 30000A | 0.91 | 7200 | 1.0% |
| 2 | CEKOL 30000A | 0.91 | 21800 | 0.1% |
| 3 | CEKOL 2000S | 1.26 | 21200 | 1.0% |
| 4 | CEKOL 2000S | 1.26 | 50500 | 0.1% |
| 5 | CEKOL 50000 | 0.60 | 28000 | 0.1% |
| 6 | CEKOL 30000 | 0.92 | 19600 | 0.1% |

2. Solution Preparation (Hydrocolloid Dissolution Rate)

An important quality of films is how quickly they dissolve or disperse. To compare the modified CMC with other hydrocolloids the solubility of hydrocolloids was compared. Solutions were prepared to a concentration that can be applied to cast films from without addition of plasticizer or other ingredients. The solutions were prepared in standardized tap water (1 g NaCl+0.219 g CaCl₂.6H₂O in 1 liter demineralised water). With an IKA Viscoklick system attached to an upper stirrer the torque was monitored. At the time that a hydrocolloid is in solution, the torque becomes constant (constant viscosity). This time to constant torque is taken as solubility time. The table shows the hydrocolloid, the concentration of the solution prepared and solubility time. The concentration divided by the solubility time is a measure of how much hydrocolloid can be dissolved per time unit. This shows that the amount of modified CMC that can be dissolved per time unit is much higher than most other hydrocolloids, as well as the total amount of modified CMC that can be dissolved. Thus, it was believed that such modified CMC materials would provide excellent quick dissolve film components. It is important to note that although the Methocel® E5 sample exhibits excellent dissolution rates, films prepared with such materials exhibit excessive adhesion characteristics and thus, in actuality, such films would exhibit much slower dissolution in practice than modified CMC films. As noted below, the modified CMC materials exhibited much better low adhesion properties and thus in practice provided much better quick dissolution capabilities than such hydroxypropylmethylcellulose materials.

Table 2 as follows shows comparatives results of solubility of modified CMC and other hydrocolloids:

TABLE 2
Comparisons of solubility of modified
CMC with Other Hydrocolloids

| | Concentration | Solubility | Concentration/ |
|---|---|---|---|
| Hydrocolloid | of solution (%) | time (min) | solubility time |
| Example Number 2 | 40 | 9.5 | 4.21 |
| from Table 1 | | | |
| Example Number 4 | 40 | 7.6 | 5.26 |
| from Table 1 | | | |
| CMC (CPKelco, | 12 | 19.9 | 0.60 |
| Cekol ® 30) | | | |
| Pectin D slow set Z ® | 8 | 11.5 | 0.70 |
| Pectin X-939-04 ® | 12.5 | 8.7 | 1.44 |
| Radiated Cekol 30 | 20 | 9.1 | 2.20 |
| (27 kGy) | | | |
| Pullulan | 25 | 12.1 | 2.07 |
| Hydroxy propyl methyl | 15 | 16.9 | 0.89 |
| cellulose (Fluka; 15 mPas, | | | |
| 2% water at 25° C.) | | | |
| Methocel ® E5 | 30 | 3.8 | 7.9 |
| (HPMC Dow Chemicals) | | | |
| Methocel E50 | 20 | 6.9 | 2.9 |
| (HPMC Dow Chemicals) | | | |
| Keltrol ® | 3 | 7.5 | 0.40 |
| (xanthan, CPKelco) | | | |

Thus, the modified CMC types exhibited excellent solubility times and a high concentration of the modified CMC can be prepared as compared with the other hydrocolloids tested.

3. Modified CMC Film Production

The modified CMC materials from Table 1, above, were then utilized to form films in accordance with the following method: The modified CMC was weighed out and dissolved into tap water. After the modified CMC was dissolved completely, glycerol was weighed out and added to the dissolved modified CMC solution. (preferred; could be premix, too) Air bubbles within the resultant solution were removed by centrifugation or by vacuum. That solution was then cast using a draw-down bar on a plastic sheet into thin even layers. The layers were then dried at room temperature to form films exhibiting final thicknesses of between 20 and 500 μm. Table 3, below, thus indicates the different films produced with the plasticizer (i.e., glycerol) to modified CMC ratio. Note that the remainder of the solution utilized to form the films was tap water (thus, if 50% was CMC, and the plasticizer:CMC ratio is 1:10, then 5% of the solution was plasticizer, and 45% was then tap water, for example). Also, if no plasticizer was added, the term “None” is used and thus the remainder of the film-producing solution was tap water alone. Additionally, film example 18 included 6% (ratio CMC: modified CMC˜1:6) of non-modified CMC (CEKOL® 30) in combination with the noted modified type, and thus the amount of tap water was adjusted accordingly. Furthermore, film example 23 included 1% of pectin GENU® X-934-04) in combination with the noted modified type, with 22.8 g of water. Lastly, the notation of G after the plasticizer:CMC ratio denotes glycerol as the plasticizer, whereas the notation of S denotes the utilization of sorbitol.

TABLE 3
Films Produced from Modified CMC Materials

| | | | |
|---|---|---|---|
| | CMC Ex. # | (%) Plasticizer: | Thickness |
| Film Ex. # | from Table 1 | CMC Ratio | (mm) |

| 1 | 1 (50%) | 1:10 | G | 0.087 |
|---|---|---|---|---|
| 2 | 2 (35%) | 1:10 | G | 0.088 |
| 3 | 1 (45%) | 1:3 | G | 0.057 |
| 4 | 2 (35%) | 1:3 | G | 0.081 |
| 5 | 2 (35%) | None | | 0.076 |
| 6 | 3 (38.7%) | None | | 0.081 |
| 7 | 3 (40%) | 1:10 | G | 0.032 |
| 8 | 3 (40%) | 1:10 | G | 0.143 |
| 9 | 3 (40%) | 1:10 | G | 0.341 |
| 10 | 4 (40%) | 1:10 | G | 0.061 |
| 11 | 4 (40%) | 1:10 | G | 0.195 |
| 12 | 4 (40%) | 1:10 | G | 0.454 |
| 13 | 2 (40%) | 1:10 | G | 0.032 |
| 14 | 2 (40%) | 1:10 | G | 0.080 |
| 15 | 2 (40%) | 1:10 | G | 0.144 |
| 16 | 2 (40%) | 1:10 | G | 0.459 |
| 17 | 2 (40%) | 1:10 | S | 0.094 |
| 18 | 6 (35%) | None | | 0.086 |
| 19 | 6 (35%) | None | | 0.068 |
| 20 | 4 (35%) | 1:10 | G | 0.088 |
| 21 | 3 (38.7%) | 1:10 | G | 0.073 |
| 22 | 3 (40%) | 1:10 | S | 0.070 |
| 23 | 1 (40%) | 1:3 | G | 0.087 |

These resultant films were then analyzed for various physical characteristics as noted below. Note that not all of the films produced within the Table 3 above were analysed using each method below.

4. Analysis of Films

i) Flexibility

Film Examples 1-4 from Table 3 were tested for flexibility. The films produced thereby were bent backward length-wise (hairpin bending) to investigate the breaking point thereof. If the film exhibiting cracking when bent in such a fashion, it was considered a failure. Film Example numbers 2-4 exhibited no cracking. Film Example 1 exhibited greater brittleness. Film Examples 3 and 4 exhibited greater flexibility overall, but due to the high plasticizer content the films are tacky. Thus, in terms of molecular weight, at least, the higher the molecular weight, coupled with lower amounts of plasticizer provided excellent flexibility results without tackiness.

Additionally, the degree of substitution was considered as a potential influence on the flexibility of the inventive films. Film Example numbers 5 and 6 were thus tested for cracks after drying and the ability to hairpin bend as above. Film Example 6 was the better of the two, with Film Example number 5 exhibiting some cracking. It is evident from the results that a higher DS permits creation of a film of greater flexibility.

Lastly, Film Example numbers 18 and 19 (18 again including non-modified CMC) were tested for flexibility. Number 18 was better in terms of low cracking, but number 19 was effective to a lesser degree in bending.

ii) Film Dissolution

Films were cut into pieces and placed into dia frames (24×36 mm). The dia frames, containing the films, were placed into a water bath exhibiting a temperature of 37° C. The water was gently stirred and dissolution time of the films was measured in terms of monitoring by visual observation. The following Table 4 shows dissolution times (average of two separate measurements) and takes into account differences in molecular weight, degree of substitution, and film thickness as factors in film dissolution for inventive modified CMC films.

TABLE 4
Influence of DS and Mw on dissolution
time of modified CMC films

| Film Example | Thickness | | Mw | Dissolution |
|---|---|---|---|---|
| Number | (mm) | DS | (Dalton) | time (sec) |
| 7 | 0.032 | 1.26 | 21,200 | 1 |
| 8 | 0.143 | 1.26 | 21,200 | 10 |
| 9 | 0.341 | 1.26 | 21,200 | 54 |
| 10 | 0.061 | 1.26 | 50,500 | 12 |
| 11 | 0.195 | 1.26 | 50,500 | 38 |
| 12 | 0.454 | 1.26 | 50,500 | 114 |
| 13 | 0.032 | 0.91 | 21,800 | 2 |
| 14 | 0.080 | 0.91 | 21,800 | 6 |
| 15 | 0.144 | 0.91 | 21,800 | 14 |
| 16 | 0.459 | 0.91 | 21,800 | 126 |

The results show that dissolution rate increases clearly as the thickness of the films increase, an increase in molecular weight results in an increase in dissolution time, and a lower DS results in an increase in dissolution time. Thus, it was determined that all three factors accord some degree of influence on the film dissolution rate.

Lastly, further comparisons between modified CMC films and typical hydrocolloids used for making films were made as well. The following Table 5 provides those measurements. The thickness of the films is also included in the table, because thickness has a clear influence (thicker films result in longer dissolution time). The results clearly show that pullulan and the hydroxypropyl methyl cellulose films dissolve much slower than the modified CMC samples. A remark on pullulan needs to be made in that a film with only pullulan as a hydrocolloid adheres to the sheet that it has been cast on. Such a film with regular film thickness cannot be removed from the sheet. The pectin films also dissolve slower than the modified CMC films, especially when film thickness is taken into account.

TABLE 5
Dissolution times of modified CMC films
compared to other hydrocolloids

| | Plasticizer ratio | | |
|---|---|---|---|
| | hydrocolloid/ | Thickness | Dissolution |
| Hydrocolloid | glycerol | (mm) | time (sec) |
| Example # 4 from Table 1; | 10:1 | 0.061 | 12 |
| DS 1.26; Mw 50,500 | | | |
| Example # 2 from Table 1; | 10:1 | 0.080 | 6 |
| DS 0.91; Mw 21,800 | | | |
| CMC (CPKelco, Cekol ® 30) | 10:1 | 0.046 | 9 |
| Radiated CMC [radiated | 10:1 | 0.096 | 40 |
| Cekol 30 (27 kGy)] | | | |
| Pectin (CPKelco X-939-04 ®) | 10:1 | 0.023 | 14 |
| Pectin D Slow set-Z ® | 10:1 | 0.018 | 14 |
| (CPKelco) | | | |
| Hydroxypropyl methyl | 10:1 | 0.050 | 26 |
| cellulose (Fluka; 15 mPas, | | | |
| 2% water at 25° C.) | | | |
| Methocel ® E5 (HPMC Dow | 10:1 | 0.080 | 50 |
| Chemicals) | | | |
| Methocel E50 (HPMC Dow | 10:1 | 0.080 | 70 |
| Chemicals) | | | |
| Pullulan | 10:1 | 0.256 | 84 |

Thus, the modified CMC films measured provide excellent dissolution in comparison with all of these standard types.

iii) Mechanical Properties

Certain properties, such as tensile strength, elongation, toughness, and elastic modulus were measured for resultant films as well to indicate the viability of such films as potential commercial products. Such measurements were taken through standard techniques. A texture analyzer from Stable Micro Systems equipped with tensile grips was used to determine the mechanical properties of the films at 50% RH. To determine the influence of molecular weight, Film Example numbers 20 and 21 were analysed for such mechanical properties. Film Example 20 has a higher molecular weight than Film Example 21 (MW of 50500 versus 21200). The average of 6 measurements was calculated and shown in table 6.

TABLE 6
Influence molecular weight of modified
CMC on mechanical properties of films

| | Film 20 | Film 21 |
|---|---|---|
| Tensile Strength (N/mm²) | 29.4 | 13.6 |
| Elongation (%) | 6.6 | 5.2 |
| Toughness (N/mm²*%) | 144.5 | 50 |
| E-modulus (N/mm²/%) | 11.7 | 6.3 |

The toughness of Film 20 is almost 3 times as high as Film 21, while the elongation of film 20 is only 1.3% bigger than the elongation for film 21. The E-modulus and the tensile strength of Film 20 is about twice as high as for Film 21.

iv) Clarity and Haziness

Films prepared from modified CMC have a high clarity and low haziness. This is already visible when the solutions are prepared. Modified CMC was compared to other hydrocolloids used for making films. The clarity and haziness were measured with a BYK-Gardner haze-gard plus of 10% solutions of hydrocolloids. If the DS of the modified CMC is not too low the clarity is high and the haziness is low. Other hydrocolloids may have a high clarity, but they can have a high haziness like the pectin samples. The results are shown in the table below. Each sample solution mentioned below was measured at the same thickness (2 mm).

TABLE 7
Clarity and haziness of hydrocolloids solutions

| Hydrocolloid | Clarity | Haze |
|---|---|---|
| Example # 5 from Table 1; | 31.1 | 60.5 |
| Mw 28000; DS 0.60 | | |
| Example # 2 from Table 1; | 96.0 | 6.26 |
| Mw 21800; DS 0.91 | | |
| Example # 4 from Table 1; | 95.3 | 10.2 |
| Mw 50500; DS 1.26 | | |
| CMC (CPKelco, Cekol 30) | 76.5 | 15.5 |
| Radiated CMC (radiated Cekol 30 | 90.4 | 7.42 |
| (27 kGy)) | | |
| Pectin (CPKelco X-939-04) | 95.1 | 93.4 |
| Pectin D Slow set-Z (CPKelco) | 90.9 | 89.1 |
| Pullulan | 98.5 | 20.8 |
| Hydroxypropyl methyl | 98.7 | 1.94 |
| cellulose (Fluka; 15 mPas, | | |
| 2% water at 25° C.) | | |

Hence, the modified CMC films, having not too low DS levels, exhibited excellent measurements in both properties as opposed to the comparatives (MHPC is an exception).

v) Plasticizer Modifications

Other plasticizers than glycerol can be used to prepare films from modified CMC. Film Example numbers 17 and 22 prepared with sorbitol as a plasticizer resulted both in flexible and not tacky films tested in humidity range from 20% up to 70% relative humidity (RH).

5. Films with Additives

i)Confectionary Film

| Formulation | Percent |
|---|---|
| Modified CMC (example # 2) | 34 |
| Deionized Water | 54.5 |
| Glycerol (99.0%) | 1 |
| Orange Flavor (McCormick Juice type n/a OS) | 6 |
| Citric Acid | 2 |
| Malic Acid | 2 |
| Sucralose, micronized (Splenda ® Brand) | 0.5 |
| Red # 40 FD&C (10% Solution) | T.S. |
| Yellow #5 FD&C (1% Solution) | T.S. |
| Total | 100 |

Such a film was produced in accordance with the composition of the preceding table through the following process: Modified CMC was added to the water and glycerin while mixing at 1200 rpm with a propeller mixer. After addition of the modified CMC the mixing was continued at high speed. After ˜15 minutes the sucralose was added. Addition of citric and malic acid was started when the sucralose had fully dispersed. After all the acid was added first the flavor was added and then the color. When the sample was uniform in appearance, the mixer was removed and the sample was deaerated using either vacuum or centrifugation. A portion of the solution was poured on to the plastic sheet and a draw down bar was used to draw the solution down into a thin layer resulting in a film of a thickness of about 0.05 mm. The films were then allowed to stand undisturbed until thoroughly dried. The resultant films exhibited excellent dissolution times (on par with those presented above) and thus effective delivery of flavoring.

ii) Surfactant Film

| Formulation | Percent |
|---|---|
| Modified CMC example # 2) | 30 |
| Deionized Water | 49 |
| Glycerol (99.0%) | 1 |
| Sodium Lauryl Sulfate (SLS) | 20 |
| Fragrance | T.S. |
| Color | T.S. |
| Total | 100 |

Such a film was produced in accordance with the composition of the preceding table and in accordance with the following method: Modified CMC was added to the water and glycerin while mixing at 1200 rpm with a propeller mixer. After addition of the modified CMC the mixing was continued at high speed. After ˜15 minutes the remaining ingredients were added and when the sample was uniform in appearance the mixer was removed and the sample was deaerated using either vacuum or centrifugation. A portion of the solution was poured on to the plastic sheet and a draw down bar was used to draw the solution down into a thin layer of a thickness of about 0.01 inch. The films were allowed to stand undisturbed until thoroughly dried. Final film thickness was 0.002 inch. The resultant films exhibited excellent dissolution times (on par, again, with those presented above) and surfactant delivery capability.

While the invention will be described and disclosed in connection with certain preferred embodiments and practices, it is in no way intended to limit the invention to those specific embodiments, rather it is intended to cover equivalent structures structural equivalents and all alternative embodiments and modifications as may be defined by the scope of the appended claims and equivalence thereto.

What is claimed:

1. A film and/or capsule comprising modified CMC materials exhibiting a molecular weight range of from 1,500 to 75,000 Daltons and a degree of substitution of less than about 1.5; wherein said film and/or capsule optionally comprises at least one polymeric additive other than said modified CMC materials.

2. The film and/or capsule of claim 1 wherein said modified CMC materials exhibit a molecular weight range of from 7,000 to 55,000 Daltons and a degree of substitution of between 0.6 and 1.3.

3. The film and/or capsule of claim 2 wherein said modified CMC materials exhibit a molecular weight range of from 21,000 to 55,000 Daltons and a degree of substitution of between 0.9 and 1.3.

4. The film and/or capsule of claim 1 wherein said optional polymeric additive is present.

5. The film and/or capsule of claim 2 wherein said optional polymeric additive is present.

6. The film and/or capsule of claim 3 wherein said optional polymeric additive is present.

7. The film and/or capsule of claim 4 wherein said optional polymeric additive is a gel forming material present in an amount of from 0.05 to 50% by weight of the entire film and/or capsule, wherein said material is selected from the group consisting of cellulose ethers, biogums, hydrocolloids, and any mixtures thereof.

8. The film and/or capsule of claim 5 wherein said optional polymeric additive is a gel forming material present in an amount of from 0.05 to 50% by weight of the entire film and/or capsule, wherein said material is selected from the group consisting of cellulose ethers, biogums, hydrocolloids, and any mixtures thereof.

9. The film and/or capsule of claim 6 wherein said optional polymeric additive is a gel forming material present in an amount of from 0.05 to 50% by weight of the entire film and/or capsule, wherein said material is selected from the group consisting of cellulose ethers, biogums, hydrocolloids, and any mixtures thereof.

10. The film and/or capsule of claim 4 wherein said optional polymeric additive is a non-gelling viscosity building material present in an amount of from 0.05 to 50% by weight of the entire film and/or capsule, wherein said material is selected from the group consisting of cellulose ethers, biogums, hydrocolloids, and any mixtures thereof.

11. The film and/or capsule of claim 5 wherein said optional polymeric additive is a non-gelling viscosity building material present in an amount of from 0.05 to 50% by weight of the entire film and/or capsule, wherein said material is selected from the group consisting of cellulose ethers, biogums, hydrocolloids, and any mixtures thereof.

12. The film and/or capsule of claim 6 wherein said optional polymeric additive is a non-gelling viscosity building material present in an amount of from 0.05 to 50% by weight of the entire film and/or capsule, wherein said material is selected from the group consisting of cellulose ethers, biogums, hydrocolloids, and any mixtures thereof.
